(19) United States
(12) Patent Application Publication
(10) Pub. No.: US 2009/0088714 A1
(43) Pub. Date: Apr. 2, 2009

(54) Sanitary napkin

(76) Inventors: Jung-Chi Tai, Chia-Li Town (TW); Ho-Hsi Yang, Chia-Li Town (TW); Chien-Chung Su, Chia-Li Town (TW)

Correspondence Address: MEREK, BLACKMON & VOORHEES, LLC, 673 S. WASHINGTON ST, ALEXANDRIA, VA, 22314, US

(21) Appl. No.: 12/000,023

(22) Filed: Dec. 7, 2007

(30) Foreign Application Priority Data
Sep. 27, 2007 (TW) 096135955

(51) Int. Cl. A61F 13/15

(52) U.S. Cl. 604/385.03; 604/385.01; 604/385.17

(57) ABSTRACT

A sanitary napkin includes a flexible absorbing rod body, an elastic band, and a flap. The absorbing rod body has a longitudinal first side adapted to block a vaginal opening, and a longitudinal second side opposite to the first side. The elastic band is disposed longitudinally at the second side of the absorbing rod body, and applies a contracting force to the second side so that the first side is curved. The flap projects from the second side of the absorbing rod body, and is adapted to facilitate handling of the sanitary napkin.

### CROSS-REFERENCE TO RELATED APPLICATION

This application claims priority of Taiwanese Application No. 096135955, filed on Sep. 27, 2007.

### BACKGROUND OF THE INVENTION

1. Field of the Invention

This invention relates to a sanitary napkin, more particularly to a sanitary napkin that is suitable for use when wearing a thong garment.

2. Description of the Related Art

Women usually use a sanitary panty shield before or after a menstrual period or on days when a small amount of discharge or excretion occurs. However, when a user wears a thong garment, such as a T-back or G-string, or the like, and uses the aforementioned sheet-like panty shield which has a large area, the panty shield is likely to extend past the undergarment and be exposed, and is thus not suitable for use on such a garment.

### SUMMARY OF THE INVENTION

Therefore, the object of the present invention is to provide a sanitary napkin that can be disposed on an outer portion of a vaginal opening and that has a small volume so as not to extend past an undergarment of a user and thereby be exposed.

According to this invention, a sanitary napkin comprises a flexible absorbing rod body, an elastic band, and a flap. The absorbing rod body has a longitudinal first side adapted to block a vaginal opening, and a longitudinal second side opposite to the first side. The elastic band is disposed longitudinally at the second side of the absorbing rod body, and applies a contracting force to the second side so that the first side is curved. The flap projects from the second side of the absorbing rod body, and is adapted to facilitate handling of the sanitary napkin.

### BRIEF DESCRIPTION OF THE DRAWINGS

Other features and advantages of the present invention will become apparent in the following detailed description of the preferred embodiment of the invention, with reference to the accompanying drawings, in which:

FIG. 1 is a perspective view of a sanitary napkin according to the preferred embodiment of the present invention;

FIG. 2 is a sectional view of the preferred embodiment;

FIG. 3 is a schematic side view of the preferred embodiment in a state of use; and

FIG. 4 is a sectional view of the preferred embodiment in a state of use.

### DETAILED DESCRIPTION OF THE PREFERRED EMBODIMENT

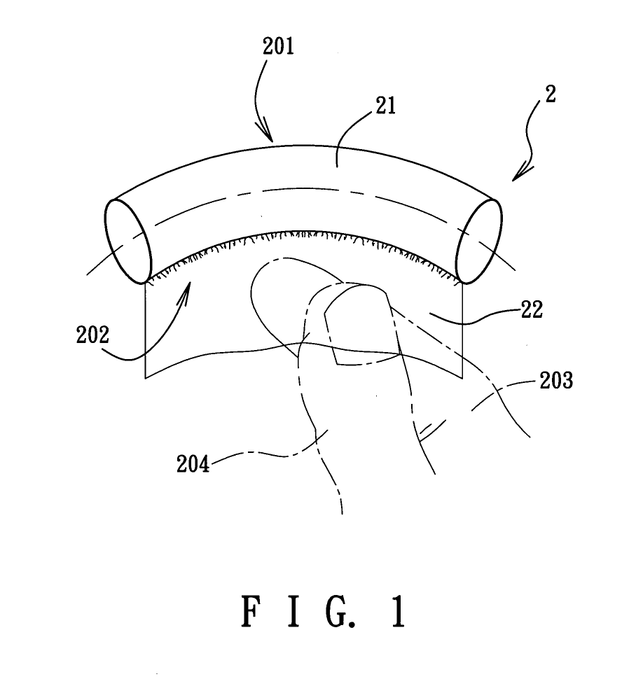

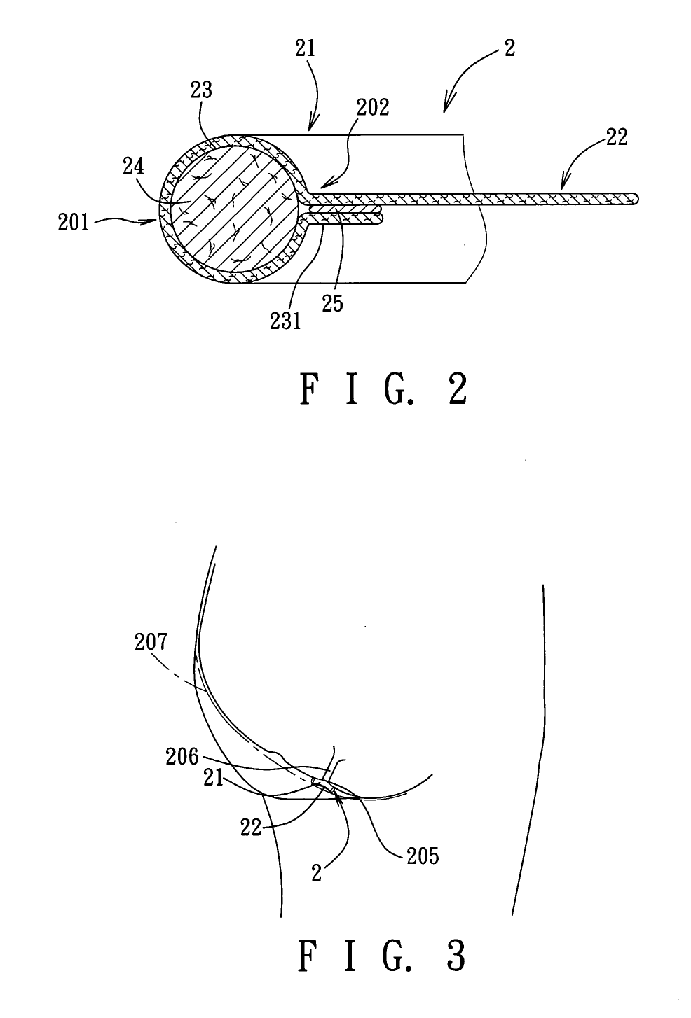

Referring to FIGS. 1 to 4, the preferred embodiment of a sanitary napkin 2 according to the present invention is shown to comprise a flexible absorbing rod body 21, a flap 22, and an elastic band 25.

The flexible absorbing rod body 21 has a circular cross-section, a longitudinal first side 201 adapted to block a vaginal opening, and a longitudinal second side 202 opposite to the first side 201. The absorbing rod body 21 includes an absorbing core 24 extending along the length of the absorbing rod body 21, and an outer layer 23 enclosing the absorbing core 24. The absorbing core 24 may be made of a material that is commonly used in an outer layer of a conventional sanitary napkin. The outer layer 23 is made of a non-woven fabric material, and has a longitudinal marginal section 231 extending at the second side 202 of the absorbing rod body 21.

The flap 22 projects from the second side 202 of the absorbing rod body 21, and is connected integrally to the outer layer 23. The flap 22 is adapted to facilitate handling of the sanitary napkin 2.

The elastic band 25 is disposed longitudinally at the second side 202 of the absorbing rod body 21, and is bonded to and is sandwiched between the marginal section 231 of the outer layer 23 and the flap 22 by using an adhesive. The elastic band 25 applies a contracting force to the second side 202, so that the first side 201 of the absorbing rod body 21 is curved. Alternatively, the elastic band 25 may be bonded to the absorbing rod body 21 using other methods, such as a hot-pressing process, an ultrasonic wave process, a high-frequency sealing process, etc.

With reference to FIGS. 1 and 3, to use the sanitary napkin 2, the user grasps the flap 22 with her fingers 203, 204, and places the first side 201 of the absorbing rod body 21 on an outer portion 205 of her vagina, so that the absorbing rod body 21 is disposed between the labia and blocks the vaginal opening 206. The flap 22 is distal from the vaginal opening 206 at this time. Since the first side 201 curves downwardly due to an applied contraction force of the elastic band 25, the first side 201 contacts more fittingly the outer portion 205 of the vagina. To remove the sanitary napkin 2, the user simply uses her fingers 203, 204 to pull away the sanitary napkin 2 from the outer portion 205 of her vagina through the flap 22.

Additionally, the outer layer 23 may undergo an embossment process so as to soften its material, so that when the absorbing rod body 21 is placed on the outer portion 205 of the vagina, the outer layer 23 does not frictionally rub against the skin of the vaginal area of the user.

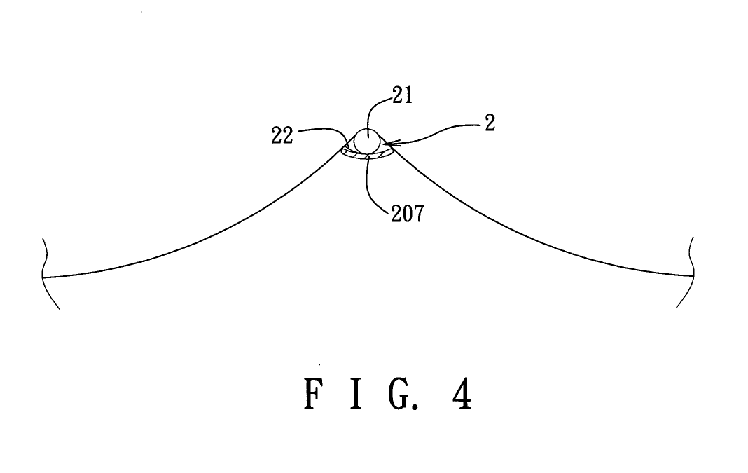

With reference to FIGS. 3 and 4, since the volume of the entire sanitary napkin 2 is small, the sanitary napkin 2 is not easily exposed from the undergarment, even when the user wears a thong garment 207.

From the aforementioned description, since the absorbing rod body 21 of the sanitary napkin 2 is located between the labia and blocks the vaginal opening 206 so as to absorb the discharge or excretion from the vagina, the sanitary napkin 2 is easy to use and remove. Further, since the sanitary napkin 2 is disposed on the outer portion 205 of the vagina, and has a small volume, as compared to the conventional sanitary panty shield, even if the user wears a thong garment 207, the sanitary napkin 2 is not likely to extend beyond the garment 207.

While the present invention has been described in connection with what is considered the most practical and preferred embodiment, it is understood that this invention is not limited to the disclosed embodiment but is intended to cover various arrangements included within the spirit and scope of the broadest interpretations and equivalent arrangements.

We claim:

1. A sanitary napkin, comprising:
a flexible absorbing rod body having a longitudinal first side adapted to block a vaginal opening, and a longitudinal second side opposite to said first side;
an elastic band disposed longitudinally at said second side of said absorbing rod body and applying a contracting force to said second side so that said first side is curved; and
a flap projecting from said second side of said absorbing rod body and adapted to facilitate handling of said sanitary napkin.

2. The sanitary napkin of claim 1, wherein said absorbing rod body includes an absorbing core extending along the length of the absorbing rod body, and an outer layer enclosing said absorbing core.

3. The sanitary napkin of claim 2, wherein said outer layer extends around said absorbing core and is connected to said flap at said second side, said elastic band being attached to said flap in close proximity to said second side.

4. The sanitary napkin of claim 3, wherein said outer layer has a longitudinal marginal section extending at said second side and bonded to said elastic band opposite to said flap, said elastic band being sandwiched between said marginal section and said flap.

5. The sanitary napkin of claim 2, wherein said outer layer is made of a non-woven fabric material.
